SEQUENCE LISTING (1 sequence)

SEQ ID NO: 1 - DNA, 1500 bp, Pseudomonas
tggagagtttgatcctggctcagattgaacgctggcggcaggcctaacacatgcaagtcgagcggcagcacgggtacttgtacctggtggcgagcggcggacgggtgagtaatgcctaggaatctgcctagtagtgggggataacgtccggaaacgggcgctaataccgcatacgtcctacgggagaaagtgggggatcttcggacctcacgctattagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagttacctaatacgtgattgttttgacgttaccgacagaataagcaccggctaactctgtgccagcagccgcggtaatacagagggtgcaagcgttaatcggaattactgggcgtaaagcgcgcgtaggtggtttgttaagttggatgtgaaagccccgggctcaacctgggaactgcatccaaaactggcaagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggta

(19) United States
(12) Patent Application Publication
(10) Pub. No.: US 2010/0093538 A1
(43) Pub. Date: Apr. 15, 2010

(54) Pseudomonas Bacterium

(75) Inventor: Samuel S. Gnanamanickam, Roanoke, VA (US)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/569,242

(22) Filed: Sep. 29, 2009

Related U.S. Application Data
(60) Provisional application No. 61/100,904, filed on Sep. 29, 2008.

(51) Int. Cl. A01N 63/02; C12N 1/20

(52) U.S. Cl. 504/117; 435/253.3

(57) ABSTRACT

According to the present invention a new isolate of a Pseudomonas spp, DSM 21663, has been shown to possess unique properties. This Pseudomonas is a plant growth-promoting rhizobacterium (PGPR). Among its modes of action involved in plant growth-promotion are anti-biotic production (2,4-diacetylphloroglucinol, (DAPG); pyrrolnitrin, PRN and others), indole-3-acetic acid (IAA) production and phosphate solubilization, and production of unique volatiles. The strain is fluorescent, oxidase-positive, and has the ability to suppress soil-borne root and foliar pathogens of both fungal and bacterial origin.

### REFERENCE TO SEQUENCE LISTING

This application contains a Sequence Listing in computer readable form. The computer readable form is incorporated herein by reference.

### REFERENCE TO A DEPOSIT OF BIOLOGICAL MATERIAL

This application contains a reference to a deposit of biological material, which deposit is incorporated herein by reference. For complete information see last page of the description.

### FIELD OF THE INVENTION

The present invention relates to an isolated Pseudomonas bacterium, and a method of promoting plant growth.

### BACKGROUND OF THE INVENTION

In order to maintain healthy growth, plants must extract a variety of elements from the soil in which they grow. These elements include phosphorus and the so-called micro-nutrients (e.g. copper, iron and zinc), but many soils are deficient in such elements or they contain them only in forms which cannot be readily taken up by plants (it is generally believed that essential elements cannot be readily taken up by plants unless they are present in dissolved form in the soil).

To counteract such deficiencies, sources of the deficient elements are commonly applied to soils in order to improve growth rates and yields obtained from crop plants. For example, phosphates are often added to soil to counteract a lack of available phosphorus. Phosphate added to the soil as a commercial fertilizer (e.g., mono-ammonium phosphate or triple-super-phosphate) is readily plant available, but is rapidly converted in soil to relatively unavailable forms. It has been estimated that only 10 to 30% of phosphate fertilizer is used by the plant in the year it is applied, and one-third to one-half of the phosphate fertilizer applied may never be recovered by the plant.

Attempts have been made in the past to use microorganisms to improve the availability of essential elements in soil systems. Kucey (Kucey, R M N. 1987. Increased phosphorus uptake by wheat and field bean inoculated with phosphorus-solubilizing Penicillium bilaji strain and with vesicular arbuscular mycorrhizal fungi. Appl. Environ. Microbiolo. 53, 2699-2703; Kucey, R M N 1988. Effect of Penicillium bilaji on the solubility and uptake of P and micro-nutrients from soil by wheat. Can. J. Soil. Sci. 68, 261-270; U.S. Pat. No. 5,026,417) describes the use of Penicillium bilaiae for improving the uptake of phosphorus by plants when applied to soil.

Phosphate solubilization by members of plant growth-promoting rhizobacteria (PGPR) has received a lot of attention in recent years (Velazquez, E and Rodriguez-Barueco (eds). 2007. Proc. of First International Meeting on Microbial Phosphate Solubilization, Springer, The Netherlands, p 361). Such phosphate-solubilizing bacteria (PSB) can be used as natural fertilizers for the development of sustainable agriculture. P is present in soil as organic and inorganic phosphates. A large portion is present in insoluble forms, and therefore not available for plant nutrition. The ability to convert insoluble phosphates to a form accessible to the plants, like orthophosphate, is an important trait for a PGPR and it can result in increased plant growth/yields. In PSB, mineral phosphate solubilizing capacity has been shown to be related to the production of organic acid (Rodriguez, H. and Fraga, R. 1999. Phosphate solubilizing bacteria and their role in plant growth-promotion. Biotechnol. Adv. 17: 319-339) such as gluconic acid (Goldstein, A. H. 1996. Involvement of the quinoprotein glucose dehydrogenase in the solubilization of exogenous phosphates by Gram-negative bacteria. In Phosphate in microorganisms: cellular and molecular biology. Eds. A. Torriani Gorini, E. Yagil and S. Silver, ASM Press, Washington, D.C., 197-203 pp).

Among the PSB, bacilli, rhizobia and pseudomonads are the most studied P-solubilizers (Rodriguez and Fraga, supra). In the current genus Pseudomonas, only P. putida, P. aeruginosa, P. corrugata, P. fluorescens, and P. stutzeri are the most studied (Martin, L., Velasquez, E., Rivas, R., Mateos, P. F., Martinez-Molina, E., Rodriguez-Barueco, C, and Peix, A. 2007. Effect of inoculation with a strain of Pseudomonas fragi in the growth and phosphorus content of strawberry plants. In E. Velasquez and C. Rodriguez-Barueco (eds) First Intl Meeting on Microbial Phosphate Solubilization, Springer, The Netherlands, pp 309-315).

Another common problem affecting healthy plant growth relates to fungal and bacterial pathogens causing a reduction in yield or even plant death. Methods available for biological control of fungal pathogens on plants have included bacterial strains of the species Pseudmonas having pathogen-specific activity. U.S. Pat. No. 4,456,684 describes Pseudomonas strains which suppress disease caused by Gaeumannomyces graminis. Many of the most effective strains reported to date produce the antibiotic 2,4-diacetylphloroglucinol (Phl) (C. Keel et al., Applied and Environmental Microbiology 62:552-563 (1996)).

Involvement of 2,4-diacetylphloroglucinol, DAPG, in biological disease control and the importance of DAPG as a bio-control compound that is responsible for the suppression several globally important plant pathogens (such as Gauemannomyces tritici var. tritici, the pathogen of wheat “take-all” disease, Thievalviopsis basicola, the pathogen of tobacco black root-rot, Xanthomonas oryzae pv. oryzae, the bacterium that causes bacterial blight of rice, and others like Fusarium oxysporum, Pythium and Rhizoctonia) is well known (Stutz, E. W., Defago, G., and Kern, H. 1986. Naturally occurring fluorescent pseudomonads involved in suppression of black root rot of tobacco. Phytopathology 76: 184-185; Velusamy, P., Ebenezar Immanuel, J., Gnanamanickam, S. S and Thomashow, L. S. 2006. Biological control of rice bacterial blight by plant associated bacteria producing 2,4-diacetylphloroglucinol. Can. J. Microbiol. 52: 56-65). Production of DAPG in wheat rhizosphere is considered responsible for “take-all decline” in the Pacific North West of the United States (Cook, R. J. 2002. Preface. In Biological Control of Crop Diseases, S. S. Gnanamanickam (ed), Dekker/CRC Press, 2002). The compound has a broad-spectrum of activity against fungi, bacteria, viruses and nematodes.

Pyrrolnitrin (PRN)(3-chloro-4-[2′ nitro-3′-chlorophenyl]pyrrole) is yet another important secondary metabolite produced by different species of Pseudomonas (P. fluorescens, P. chlororaphis, P. aureofaciens) Burkholderia cepacia, B. pyrrocinia, Enterobacter agglomerans, Myxococcus fulvus and Serratia species (Burkhead, K. D., Schisler, D. A, and Slininger, P. J. 1994. Pyrrolnitrin production by biological control agent Pseudomonas cepacia B37W in culture and in colonized wounds of potato. Appl. Environ. Microbiol. 60: 2031-2039).

U.S. Pat. No. 4,647,533 reports Pseudomonas strains which suppress diseases caused by Pythium. Strains of Pseudomonas bacteria inhibitory to either Rhizoctonia solani or Pythium ultimum on cotton have been reported (See U.S. Pat. No. 5,348,742 to Howell et al.). Bacillus sp. L324-92 has been reported to simultaneously control Gaeumannomyces graminis, Rhizoctonia and Pythium species (Kim et al. Phytopathology 87:551-558 (1997)).

Among all of the Pseudomonas strains reported so far being capable of efficient disease control and phosphate solubilization most of them belong to Pseudomonas fluorescens. Other Pseudomonas spp such as P. stutzeri, P. putida, and P. fragi can also function as P-solubilizers. Pseudomonas congelans, however, isolated from the phyllosphere of grasses and described as a new species in 2003 (Behrendt, U., Ulrich, A, and Schumann, 2003. Fluorescent pseudomonads associated with the phyllosphere of grasses; Pseudomonas trivialis sp. nov., Pseudomonas poae, sp. nov., and Pseudomonas congelans sp. nov., Int. J. Syst. Evol. Microbiol. 53: 1461-1469) is not capable of phosphate solubilization and there are no reports of its promotion of plant growth, suppression of plant pathogens or production of phytotoxic and insect-toxic volatiles.

### SUMMARY OF THE INVENTION

The invention provides a novel species belonging to the Pseudomonas genus having both phosphate solubilising properties as well as plant pathogen suppressive properties.

In a first aspect the present invention provides an isolated Pseudomonas bacterium which has at least the characteristics:

i) is fluorescent;

ii) naturally encodes for the production of indophenol oxidase;

iii) naturally encodes for the production of 2,4-diacetylphloroglucinol, pyrrolnitrin, and indole-3-acetic acid;

iv) has the ability to promote solubilization of soil phosphate;

v) suppresses soil-borne plant pathogens of both fungal and bacterial origin; and

vi) has a 16S rDNA nucleic acid sequence comprising SEQ ID No. 1 or a nucleic acid sequence at least 98% identical to SEQ ID No. 1.

In a second aspect the present invention provides an isolated Pseudomonas strain, wherein the said strain is DSM 21663.

In a third aspect the present invention provides a composition comprising the isolated Pseudomonas bacterium according to the invention.

In a fourth aspect the present invention provides a method of enhancing plant growth, comprising applying to plants, plant seeds, or soil surrounding plants, or plant seeds a composition comprising the isolated Pseudomonas bacterium according to the invention.

### BRIEF DESCRIPTION OF DRAWINGS

FIG. 1 shows the phylogenetic tree of 1500 by resolution of 16S rDNA. DSM 21663 is most closely related to P. congelans, however, the distance with which it is separated from P. congelans strongly indicates that this strain is a unique species within Pseudomonas.

FIG. 2 shows the inhibitory effect of bacterial volatiles produced from DSM21663 on sclerotia germination. Mycelial growth (measured as cm) of Sclerotinia after 3 days is shown for different experiments.

FIG. 3a shows the biocontrol of Fusarium Head Blight of wheat under growth chamber conditions when DSM21663 is applied to heads. Incubation for 10 days after applying the Fusarium pathogen. Disease incidence as count of infected heads divided by total number is shown in FIG. 3a.

FIG. 3b shows the biocontrol of Fusarium Head Blight of wheat under growth chamber conditions when DSM21663 is applied to heads. Incubation for 10 days after applying the Fusarium pathogen. Infected heads severity as average of severity scores from infected heads (based on disease scale) is shown in FIG. 3b.

### DETAILED DESCRIPTION OF THE INVENTION

According to the present invention a new isolate of a Pseudomonas spp, DSM 21663, has been shown to possess unique properties. This Pseudomonas is a plant growth-promoting rhizobacterium (PGPR). Among its modes of action involved in plant growth-promotion are anti-biotic production (2,4-diacetylphloroglucinol, (DAPG); pyrrolnitrin, PRN and others), indole-3-acetic acid (IAA) production and phosphate solubilization, and production of unique volatiles. The strain is fluorescent, oxidase-positive, and has the ability to suppress soil-borne plant pathogens of both fungal and bacterial origin.

DSM 21663 was isolated from rhizosphere samples (roots with some soil) in rice fields of Kerala in Southern India during 1987-88.

In order to further characterize this isolate having a unique combination of properties as listed above a phylogenetic analysis was carried out based on a 1500 by nucleic acid sequence fragment derived from ribosomal 16S RNA. From the analysis it was concluded that the isolate belongs to the genus Pseudomonas, however, as explained in more detail in the examples it was not possible from this analysis to characterize the isolate as belonging to any known species of Pseudomonas. The analysis revealed that the isolate is most closely related to P. congelans but only by 98.6% identity. In many aspects the isolate resembles P. fluorescens, however, according to the analysis the new strain is only 97.3% identical to P. fluorescens. It therefore appears that the isolate belongs to a new species and according to the present invention the isolate has been defined by its unique properties. In one aspect the invention therefore relates to an isolated Pseudomonas bacterium which has at least the characteristics:

i) is fluorescent;

ii) naturally encodes for the production of indophenol oxidase;

iii) naturally encodes for the production of 2,4-diacetylphloroglucinol, pyrrolnitrin, and indole-3-acetic acid;

iv) has the ability to promote solubilization of soil phosphate;

v) suppresses plant pathogens of both fungal and bacterial origin; and

vi) has a 16S rDNA nucleic acid sequence comprising SEQ ID No. 1 or a nucleic acid sequence at least 98% identical to SEQ ID No. 1.

In the context of the present invention the term “naturally encodes for” means that the isolated Pseudomonas spp contains the necessary gene(s) in order to produce the said product.

Production of indophenol oxidase results in an oxidase positive phenotype. The skilled person will know how to isolate an oxidase negative mutant starting from the isolated strain according to the invention. Such a mutant strain still belongs to the Pseudomonas spp according to the invention since it will be derived from an original isolate that contains the genetic elements necessary to produce indophenol oxidase.

For purposes of the present invention, the degree of identity between two deoxyribonucleotide sequences is determined using the Kimura 2-parameter distance model which corrects for multiple hits, taking into account transitional and transversional substitution rates, while assuming that the four nucleotide frequencies are the same and that rates of substitution do not vary among sites (Nei and Kumar, 2000) as implemented in the MEGA 4 (Tamura K, Dudley J, Nei M & Kumar S (2007) MEGA4: Molecular Evolutionary Genetics Analysis (MEGA) software version 4.0. Molecular Biology and Evolution 24:1596-1599), preferably version 4.0.2 or later. The gap opening and extension penalties are set to 15 and 6.66 respectively. Terminal gaps are not penalized. The delay divergent sequences switch is set to 30. The transition weight score is set to 0.5, as a balance between a complete mismatch and a matched pair score. The DNA weight matrix used is the IUB scoring matrix where x's and n's are matches to any IUB ambiguity symbol, and all matches score 1.9, and all mismatched score 0.

In another particular embodiment the Pseudomonas spp according to the invention has a 16S rDNA nucleic acid sequence at least 98.5% identical to SEQ ID No. 1, more particularly at least 99% identical and even more particularly 99.5% identical to SEQ ID NO: 1.

Pseudomonas and especially P. fluorescens has previously been described as being capable of suppressing different plant pathogens. The new isolate according to the invention also has the ability to suppress plant pathogens. Plant pathogens include both soil borne root pathogens and foliar pathogens. According to one embodiment of the invention the plantpathogens that can be suppressed comprise the fungi Pythium, Phytophthora, Rhizoctonia, Sclerotinia, Fusarium, and Venturia and the bacteria Ralstonia, Xanthomonas, Pseudomonas and Erwinia. More particularly the plant pathogens comprise Rhizoctonia solani, Sclerotinia sclerotiorum, Fusarium oxysporum, Fusarium graminearum, Phythium aphanidermatum, Phytophthora erythroseptica, Venturia inaequalis, Xanthomonas oryzae, Xanthomonas axonopodis, Xanthomonas vesicatoria, Pseudomonas syringae, Pseudomonas phaseolicola, Pseudomonas savasanoi, Erwinia amylovora and Ralstonia solanacearum.

Compared to other Pseudomonas and Bacillus strains known in the art the new Pseudomonas strain according to the invention seems to have superior properties in terms of its Phosphorus solubilizing capabilities.

Another surprising property is the DSM 21663 strain's ability to produce gaseous volatiles with phytotoxicity towards fungi, algae, plants or insects. In one embodiment the plants are selected from the group consisting of crops and weeds. In another embodiment the insect is Drosophila.

In a particular embodiment the invention relates to the deposited strain DSM 21663. In another embodiment the invention relates to a mutant of DSM 21663 that has retained all of the features described above for DSM 21663.

The new species and in particular the deposited strain has been demonstrated to be efficient against plant pathogens in plate assays and in greenhouse experiments and therefore looks promising as a plant growth enhancing microorganism. Various forms of plant growth enhancement or promotion can be achieved. This can occur at different stages, e.g. as early as when plant growth begins from seeds or later in the life of the plant. Plant growth enhancement according to the present invention encompasses greater yield, increased quantity of seeds produced, increased percentage of seeds germinated, increased plant size, greater biomass, more and bigger fruit, earlier fruit coloration, and earlier fruit and plant maturation.

In a further aspect the present invention therefore relates to a method of enhancing plant growth, comprising applying to plants, plant seeds, or soil surrounding plants or plant seeds, a composition comprising the isolated Pseudomonas bacterium according to the invention. As a result of the application of the Pseudomonas bacterium according to the invention plant pathogens will be suppressed or inhibited.

Particularly soil-borne root and foliar pathogens are suppressed by the method of the invention.

In a further particular aspect the availability of phosphorous for plant uptake is improved by the presence of the bacterium according to the invention.

The source of phosphorous may be from a source originally present in the soil, sources added to the soil, and combinations thereof. By “source” of a particular element we mean a compound of that element which, at least in the soil conditions under consideration, does not make the element fully available for plant uptake.

In one embodiment said source is rock phosphate. In another embodiment said source is a manufactured fertilizer.

Commercially available manufactured phosphate fertilizers are of many types. Some common ones are those containing monoammonium phosphate (MAP), triple super phosphate (TSP), diammonium phosphate, ordinary superphosphate and ammonium polyphosphate. All of these fertilizers are produced by chemical processing of insoluble natural rock phosphates in large scale fertilizer-manufacturing facilities and the product is expensive. By means of the present invention it is possible to reduce the amount of these fertilizers applied to the soil while still maintaining the same amount of phosphorus uptake from the soil.

In a further particular embodiment the source or phosphorus is organic. An organic fertilizer refers to a soil amendment derived from natural sources that guarantees, at least, the minimum percentages of nitrogen, phosphate, and potash. Examples include plant and animal by-products, rock powders, seaweed, inoculants, and conditioners. These are often available at garden centers and through horticultural supply companies. In particular said organic source of phosphorus is from bone meal, meat meal, animal manure, compost, sewage sludge, or guano.

Other fertilizers, such as nitrogen sources, or other soil amendments may of course also be added to the soil at approximately the same time as the Pseudomonas bacterium or at other times, so long as the other materials are not toxic to the fungus.

Since the bacterium has the effect of solubilizing phosphates which may already be present in soil (i.e. those which are native to the soil) and also those which are added to the soil, the fungus may be applied alone to soils which contain native sources of phosphorus, or may be applied to any soils in conjunction with added sources of phosphorus. The inoculums comprising the bacterial strain according to the invention can be provided using solid state or liquid fermentation and a suitable carbon source. The skilled person in the art will know how to grow Pseudomonas bacteria.

The method of the present invention can be utilized to treat a wide variety of plants or their seeds to impart disease protection and/or to enhance growth. Suitable plants include dicots and monocots. More particularly, useful crop plants can include: alfalfa, rice, wheat, barley, rye, cotton, sunflower, peanut, corn, potato, sweet potato, bean, pea, chicory, lettuce, endive, cabbage, brussel sprout, beet, parsnip, turnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane. Examples of suitable ornamental plants are: Arabidopsis thaliana, Saintpaulia, Petunia, Pelavgonium, Euphovbia pulchevvima (poinsettia), Chrysanthemum, Dianthus caryophyllus (carnation), and Zinnia.

The method of the present invention can be carried out through a variety of procedures when all or part of the plant is treated, including leaves, stems, roots, plant products (e.g., grain, fruit, forage, crop debris), propagules (e.g. cuttings), etc. Suitable application methods include high or low pressure spraying, drenching, and injection. When treating plant seeds, in accordance with the present invention, the bacterium according to the invention can be applied by low or high pressure spraying, coating, immersion, or injection. Other suitable application procedures can be envisioned by those skilled in the art. Once treated with the bacterium of the present invention, the seeds can be planted in natural or artificial soil and cultivated using conventional procedures to produce plants. After plants have been propagated from seeds treated in accordance with the present invention, the plants may be treated with one or more applications of the bacterium of the present invention to impart disease protection to plants and/or to enhance plant growth. The bacterium can be applied to plants or plant seeds in accordance with the present invention alone or in a mixture with other materials. Alternatively, the bacterium can be applied separately to plants with other materials being applied at different times.

The amount of the inoculum to be applied to the soil is not limited in any particular respect. Clearly, if insufficient is used, a noticeable effect will not be obtained. On the other hand, the use of large amounts of the inoculum will be wasteful because the amounts of phosphorus and/or micronutrients made available in the soil reach a maximum at a certain application rate and further additions beyond this rate do not give additional benefits. The suitable application rates vary according to the type of soil, the type of crop plants, the amounts of the source of phosphorus and/or micronutrients present in the soil or added thereto, etc. and a suitable rate can be found without difficulty by simple trial and error experiments for each particular case. Normally, the application rate falls into the range of 10²-10⁶ colony forming units (cfu) per seed (when coated seeds are used), or on a granular carrier applying between 1×10⁶ and 1×10¹² colony forming units per hectare, more particularly between 1×10⁷ and 1×10¹¹ cfu/hectare. According to one embodiment of the method the Pseudomonas bacterium is applied as seedling root-dip or as soil drench at about 10⁶ to 10⁸ cfu/ml.

In a further aspect the present invention relates to a composition comprising the isolated Pseudomonas bacterium according to the invention, and a carrier. Suitable carriers include water, aqueous solutions, slurries, solids (e.g. peat, wheat, bran, vermiculite, and pasteurized soil) or dry powders. In this embodiment the composition contains 10⁶ to 10⁸ colony forming units per milliliter of carrier.

The composition may contain additional additives including buffering agents, wetting agents, coating agents, and abrading agents.

### EXAMPLES

### Example 1

### Identification of the Pseudomonas Isolate DSM 21663 by 16S rDNA Analysis

Phylogenetic comparison calculations were done using the program CLUSTALX to align a 1500 by sequence from the 16S RNA gene the, (SEQ ID NO: 1) to other closely related species (indicated by an initial BLAST analysis of the sequence). Once the multiple alignment file was created, a Neighbor Joining tree was constructed, and a distance matrix was calculated as the basis for identifying the genus and species of the strain. All alignment and phylogenetics related operations were done in Mega 4.0. Sequences were imported into the Alignment Explorer in Mega, and then aligned using ClustalW. From the analysis performed at MIDI Labs in Newark, a DNA distance matrix was obtained and a phylogenetic tree was constructed using bootstrapping to test the robustness.

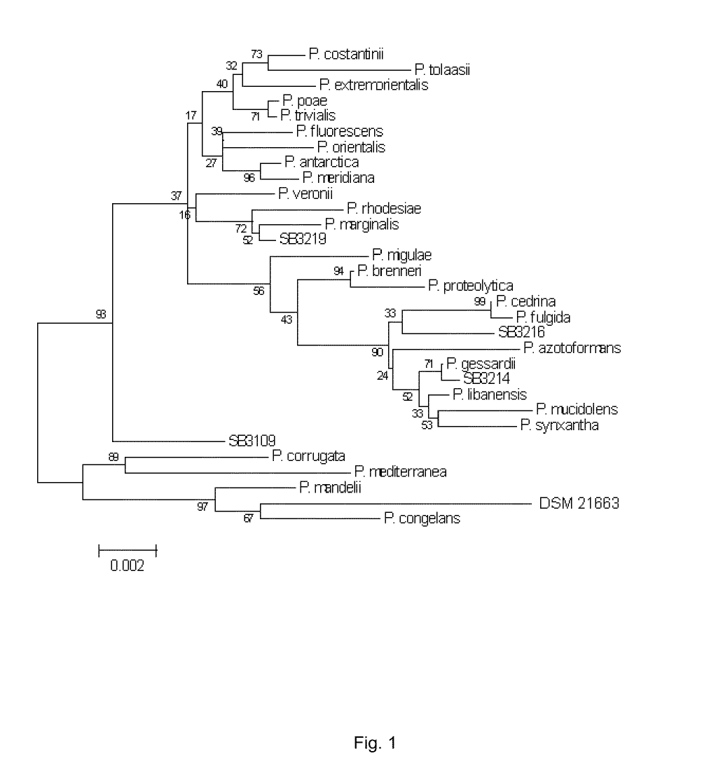

The phylogenetic tree shown in FIG. 1 represents the fluorescens branch of Pseudomonas. DSM 21663 was most closely related to Pseudomonas congelans, but only by 98.6% identity, putting it on the edge of confidence for saying that it is an isolate of this species. The strain is only 97.3% identical to Pseudomonas fluorescens. The isolate according to the invention is therefore not a Pseudomonas fluorescens and most likely not a Pseudomonas congelans either. Rather this indicates that the Pseudomonas spp. according to the invention belongs to a new species.

### DNA-DNA Hybridization of DSM 21663 to Closely Related Pseudomonas Isolates

DNA was isolated using a French pressure cell (ThermoFisher Scientific) and was purified by chromatography on hydroxyapatite as described by Cashion, et al. (1977, Anal. Biochem. 81:461-466). DNA-DNA hybridization was carried out as described by De Ley et al. (1970, Eur. J. Biochem. 12:133-142) under consideration of the modifications described by Huss, et al. (1983, Syst. Appl. Microbiol. 4:184-192) using a model Cary 100 Bio UV/VIS spectrophotometer equipped with a Peltier-thermostatted 6×6 multicell changer and a temperature controller with an in situ Varian temperature probe.

The DNA-DNA hybridization was performed comparing DNA from DSM 21663 to Pseudomonas congelans, Pseudomonas corrugata, Pseudomonas mediterranea, and Pseudomonas mandelii. As can be seen in Table 1, Pseudomonas strain DSM 21663 does neither belong to the same species as any of the other species testes, when the recommendations of a threshold value of 70% DNA-DNA similarity for the definition of a bacterial species by the ad hoc committee (Wayne, et al. 1987, Int. J. Syst. Bacteriol. 37:463-464) are considered.

TABLE 1
% DNA-DNA similarity in 2X SSC + 10% formamide at 67° C.

| | Pseudomonas DSM 21663 |
|---|---|
| Pseudomonas mandelii DSM 17967 | 39.0 (30.5) |
| Pseudomonas mediterranea DSM 16733 | 31.7 (27.8) |
| Pseudomonas corrugata DSM 7228 | 19.6 (22.9) |
| Pseudomonas congelans DSM 14939 | 18.9 (23.5) |

(values in parentheses are results of measurements in duplicate)

Physiological Characteristics of DSM 21663 Compared to Closely Related Pseudomonas species

Morphological and physiological characterization of the isolates was performed as previously described in Behrendt et al (1999, Int. J. Syst. Bacteriol. 49:297-308). As can be seen in Table 2 below, the Pseudomonas isolate DSM 21663 has specific characteristics that mark it different from the other closely related Pseudomonas species tested. This data, in addition to the DNA-DNA hybridization data, as well as the 16S rDNA sequencing data all supports the DSM 21663 strain as being a new species of Pseudomonas.

TABLE 2
Physiological characteristics of closely related Pseudomonas species.

| | DSM | P. | P. | P. | P. |
|---|---|---|---|---|---|
| | 21663 | congelans | corrugata | mediterranea | mandelii |
| Produce | + | + | − | + | + |
| fluorescent | | | | | |
| pigment | | | | | |
| Oxidase | + | − | + | + | + |
| Gelatin | + | + | + | + | + |
| Hydrolysis | | | | | |
| Arginine | + | − | − | + | + |
| Dihydrolase | | | | | |
| Utilization | | | | | |
| of: | | | | | |
| Glucose | + | + | − | + | − |
| Citrate | + | + | + | + | + |
| Melibiose | + | − | − | + | − |
| Arabinose | − | − | − | + | − |

### Example 2

### Bio-Control Plate Assay

A dual plate laboratory assays (assays for in vitro antibiosis) was carried out in order to provide an indication of the antimicrobial spectrum of a candidate bacterium/fungus. In such assays, an antagonist placed in proximity of a plant pathogen (fungus/bacterium) will inhibit the growth of the pathogen. Pathogen inhibition can be seen as a zone of inhibition around the antagonist.

DSM 21663 has been evaluated in dual plate laboratory assays against a number of plant pathogens, both fungi and bacteria.

### Dual Plate Laboratory Assay:

### Antifungal and Antibacterial Assays for Pseudomonas DSM 21663

Plant pathogenic bacterial cultures (listed in Table 4) were prepared by picking a single colony and adding to 9 mL of plate count broth (PCB). Plate count broth consists of yeast extract 5.0 g, tryptone 10.0 g and dextrose 2.0 g/liter and a final pH of 7.0-7.2. It is autoclaved at 121° C. for 15 min before use. The cultures were incubated overnight at 35° C. in a shaker.

Fungal cultures were prepared from actively growing cultures of fungi (listed in Table 3) from potato dextrose agar (PDA) plates. PDA was usually prepared from a dry powder that consists of potato starch 4.0 g, dextrose 20.0 g and agar 15.0 g/liter. The agar medium was autoclaved at 121° C. for 15 min and had a final pH of 5.6±0.2.

Sterile scalpels were used to cut 5×5 mm squares of actively growing fungal mycelium. The squares were transferred directly to the center of a fresh PDA plate and were slightly pressed down.

An overnight-grown culture of Pseudomonas DSM 21663 was diluted using PCB to adjust the cell numbers to OD of 0.2-0.4 at 600 nm. An aliquot of 7 μL of DSM 21663 bacterial suspension was placed onto plates that received the fungal mycelium as droplets in three separate spots at equal spacing closer to the rim of plate in two replicate plates. The plates were allowed to dry with lids off for 30 minutes.

The assay plates were covered in para-film and incubated at 30° C. right side up. After 5-7 days, measurements of diameter of fungal inhibition zones that had developed around each bacterial spot of DSM 21663 were taken.

Three measurements of the diameter of fungal inhibition were made for each of the three colonies of bacterial growth. The diameter of inhibition zones does not include the diameter of bacterial growth.

Results on antifungal and antibacterial spectrum are presented in the following tables (Table 3, 4):

TABLE 3
Antifungal spectrum of Pseudomonas spp DSM 21663

| Test strain | R. solani | F. oxys | Venturia | Pythium | Phytoph |
|---|---|---|---|---|---|
| DSM 21663 | +¹ | − | + | +++ | ++¹ |

Fungal pathogens tested are Rhizoctonia solani, Fusarium oxysporum, Venturia inaequalis, Pythium aphanidermatum, and Phytophthora erythroseptica.
¹Symbol + indicates inhibition (+ = 0-5 mm dia. inhibition; ++ = 5-10 mm zone and +++ = >10 mm zone)

TABLE 4
Inhibition of bacterial pathogens by Pseudomonas ssp DSM 21663

| Test Strain | Xanthomonas | P. syringae | R. solanacearum | E. amylovora |
|---|---|---|---|---|
| DSM 21663 | +¹ | + | +++ | +++¹ |

Bacterial plant pathogens tested are Xanthomonas oryzae pv. oryzae (1 strain), Xanthomonas axonopodis pv. vesicatoria (2 strains), Pseudomonas syringae pathovars (pv) phaseolicola (strain) pv. savasanoi (1 strain) and pv. syringae (2 strains), Ralstonia solanacearum (2 strains) and Erwinia amylovora (2 strains).
¹Symbol + indicates inhibition (+ = 0-5 mm dia. inhibition; ++ = 5-10 mm zone and +++ = >10 mm zone).

### Example 3

### Detection of P-Solubilization by Pseudomonas DSM 21663 in Solid and Liquid Media

### Plate Assay:

Modified Sperber's solid medium (Alikhani, Saleh-Rastin and Antoun, Proc. 1^{st} Intl Meeting on Microbial Phosphate Solubilization, 2007) was used.

The basal Sperber (1958) contained (in g/L of distilled water):

| | |
|---|---|
| Glucose | 10.0 |
| Yeast extract | 0.5 |
| CaCl₂ | 0.1 |
| MgSO₄•7H₂O | 0.25 |
| Agar | 15.0 |

The Sperber's was supplemented with 2.5 g/L of Ca₃(PO4)2 as P source. The pH was adjusted to 7.2 before autoclaving. The autoclaved medium was distributed in 9.0 cm Petri plates and marked in four equal parts after solidification.

Inoculation of bacterial strains: The four parts of each Sperber agar plate were inoculated with 7 μl of a dilute cell suspension of each test strain. Inoculated plates were incubated in darkness at 27° C.

Diameter of a clear zone (halo) that developed around bacterial growth of strains that are positive for P-solubilization was measured after 10, 20 and 30 days. Each assay was replicated 4 times and the results were recorded as the ratio of halo diameter/colony diameter (Table 3).

### P-Solubilization in Liquid Medium:

Medium NBRIP (Nautiyal, C. S., 1999. An efficient microbiological growth medium for screening phosphate-solubilizing microorganisms. FEMS Microbiol. Lett. 170: 567-572; U.S. Pat. No. 6,638,730) contained g/L:

| | |
|---|---|
| Glucose | 10.0 |
| Ca₃(PO₄)₂ | 5.00 |
| MgSO₄•7H₂O | 0.25 |
| MgCl₂•6H₂O | 5.00 |
| KCl | 0.2 |
| (NH₄)₂SO4 | 0.1 |
| Agar | 15.0 |
| Bromophenol blue | 0.025 |
| pH | 7.2 |

The NBRIP medium was prepared and dispensed into volumes of 50 ml contained in 100 ml conical flasks before autoclaving. The sterile medium was inoculated with 1.0 ml of a dilute cell suspension made from 24 hr grown plate cultures of each candidate strain. There were 3 replicates for each strain and the inoculated flasks were incubated at 27° C. on a rotary shaker at 150 rpm.

A color change from dark blue to light blue occurred in flasks inoculated with Pseudomonas DSM 21663. A change was also observed in NBRIP solid medium (that contained agar at 15.0 g/L) when inoculated with DSM 21663. Growth of DSM 21663 also corresponded to lowering of original pH 7.2 to 5.5.

TABLE 5
List of strains that were tested for P-solublization in the laboratory

| | | Dia. of halo/Dia. |
|---|---|---|
| | P-solubilization | of colony DH/CD |
| Bacterium | (+/−) | (after 20 d of growth) |
| Paenibacillus azotofixans | − | 0 |
| Bacillus licheniformis 3086 | − | 0 |
| Pseudomonas DSM 21663 | + | 2.78¹ |
| Bacillus mojavensis 3617 | − | 0 |
| Bacillus mojavensis Vc6 | − | 0 |
| Paenibacillus polymyxa AU41 | − | 0 |
| Pseudomonas strains: | | |
| P. fluorescens 3622 | + | 1.0 |
| P. stutzeri | + | 0.9 |
| P. alkaligens | − | 0.9 |

¹Among all the different bacteria tested in the laboratory, only DSM 21663 produced larger haloes that had an average of 19.5 mm in diameter after 20 days of growth on Sperber's medium.

From the plate assay it appears that DSM 21663 is superior in it ability to solubilize phosphorus. The results shown in table 5 above can be obtained using any strain of the specified genus and species and such strains may be obtained from any suitable strain collection.

### Example 4

### Analysis of DAPG and PRN Production by Pseudomonas DSM 21663

This section deals with the production, isolation and characterization of two important antimicrobial compounds produced by Pseudomonas DSM 21663.

DAPG is 2,4-diacetylphloroglucinol (a polyketide antibiotic; MW 210.18) and PRN is pyrrolnitrin. In addition to these compounds there were also one or two other antimicrobial principles that remain unidentified.

### DSM 21663.

Production of DAPG and PRN by this strain was analyzed in the laboratory by using simple methods of growth in specific media, isolation and separation using thin layer chromatography (TLC). Authentic samples of DAPG and PRN obtained either from commercial source or well known researchers were used as references in their identification through co-chromatography.

### DAPG Extraction and Analysis:

DSM 21663 was grown on malt agar (15 g malt extract, 17 g agar and 1 L double-distilled water) plates for 3 days at 28° C. The agar mats were cut into small pieces and were extracted with 250 mL of 80% aqueous acetone. The extract was filtered through Whatman No. 1 filter paper and condensed to one-third of the initial volume of acetone in-vacuo at 45° C. in a rotary evaporator (Buchler, Germany). The aqueous concentrates were acidified to pH 2 with 2N HCl and extracted three times in ethylacetate. The ethylacetate extracts were reduced to dryness in-vacuo. The residue was dissolved in 3 mL of (65%) methanol. TLC analyses of ethylacetate extracts of culture fluids were carried out by the procedure described by de Souza and Raaijmakers (2003) (de Souza, J. T and Raiijmakers, J. M. 2003. Polymorphisms within the prnD and pltC genes from pyrrolnitrin and pyoluteorin-producing Pseudomonas and Burkholderia spp. FEMS Microbiology Ecology 1454: 1-14) by applying 50 μl aliquots to pre-coated silica gel plates (20×20 cm, with fluorescent indicator F₂₅₄, Merck, Germany) and separating with a chloroform-acetone (9:1) solvent system.

DAPG purchased from TRC (Toronto Chemical Co.) served as the authentic reference. When this standard was co-chromatographed at 5-10 μg concentrations with an extract of DSM 21663, both DAPG and the bacterial extract had a uv-light absorbing compound at R_{f} (Relative distance of the distance traveled by the compound to the total distance traveled by the solvent) of 0.33. Other TLC solvent systems and bioassays were also used to confirm the presence of DAPG (Table 6).

### PRN Extraction and Analysis:

DSM 21663 was grown by shaking at 180 rpm for 24 h in 25 ml of PRN medium at 25° C. (PRN medium containing per liter: 30.0 g glycerol, 3.0 g K₂HPO₄, 5.0 g NaCl, 0.5 g MgSO₄, 7H2O, and 0.61 g D-tryptophan. The autoclaved medium was amended with filter-sterilized (0.2 μm) ZnSO₄ (0.35 mM) and MO₇(NH₄)⁶O₂₄.4H₂O (0.5 mM)), and subsequently incubated 4 days in the dark at 25° C. without shaking.

To detect PRN by TLC, 15 ml of bacterial culture was centrifuged for 15 min. at 8700×g and the cells were extracted twice with 5 ml of ethylacetate by sonicating for 3 min. The organic phase was evaporated to dryness and the residue was suspended in 150 μl of EtOAc (ethylacetate). A volume of 50 μl of this extract was applied to 0.25 mm Si gel (also containing F₂₅₄) plate (20×20 cm glass plates). PRN was detected applying (co-chromatography) 6-10 μg of PRN standard that was received from Dr. N. E. Mahoney, USDA-ARS, Albany, Calif.). The TLC was developed in chloroform-acetone (9:1) solvent. The bacterial extract had a fluorescent substance at R_{f} 0.44 which also corresponded to the R_{f} value of PRN standard. Other TLC solvent systems used to confirm the presence of PRN are listed in Table 6.

TABLE 6
R_{f} values and biological properties of secondary
metabolites produced by DSM21663

| | | | Fungal | Bacterial |
|---|---|---|---|---|
| Compound | Solvent | Rf value | inhibition¹ | inhibition² |
| 1. DAPG | Chloroform/acetone, 9:1 | 0.33 | Rhizoctonia | Xanthomonas |
| | Chloroform/methanol, 19:1 | 0.20 | solani | campestris pv. |
| | Chloroform/methanol/water | 0.78 | | pelagoni |
| | 80:15;1 | | | |
| 2. PRN | Chloroform/acetone, 9:1 | 0.44 | Rhizoctonia | Xanthomonas |
| | Chloroform, 100% | 0.30 | solani | campestris pv. |
| | Chloroform/acetone, | 80 | | pelargoni |
| | 9:1(on Si gel with | | | |
| | aluminum oxide 60 F₂₅₄) | | | |

^{1,2}Results from bioautography (Si gel was scraped and emptied into agar plates that have been previously inoculated with R. solani or Xanthomonas campestris pv. pelargoni.

### Example 5

### IAA Production by DSM 21663

EtOAC extracts of cell-free culture filtrates from selected PGPR organisms, Bacillus licheniformis 3086 as well as DSM 21663, inhibited the growth of germinating lettuce seeds when used at concentrations similar to IAA at 1 μg/ml. IAA also inhibits the germination of lettuce seeds at this concentration. When the extracts were used at 10³, 10⁴ and 10⁵ X dilutions (with IAA dilutions as reference), we observed elongation of root and shoot growth similar to those observed in IAA dilutions. This strongly suggested the production of an IAA-like plant growth hormone by PGPR strain DSM 21663.

Rapid Assay for Detection of IAA production

IAA agar and broth as described in the literature for IAA production was used. Some rapid assays for IAA production by DSM 21663 as described below were also used.

### IAA Agar:

The agar ((D-glucose-0.5%, Casamino acids-2.5%, MgSO₄.7H₂O-0.03%, K₂HPO4-0.17% and NaH₂PO4-0.2% and Difco agar, 0.7%) (Hutzinger, O and Kosuge, T. 1968. Microbial synthesis and degradation of indole-3-acetic acid. III. The isolation and characterization of indole-3-acetyl-e-L-lysine. Biochemistry (Wash.) 7: 601-605)) was amended with 1.0, 2.0, 5.0 mM tryptophan. In separate plates amended with or without L-tryptophan, bacterial strains were tooth-picked (9 spots/plate). This was followed immediately by overlaying each inoculated plate with a nitrocellulose membrane (85 mm diameter). The plates were incubated with the membrane for 3 days. At the end of the incubation period, the membranes were removed from the plates and were overlaid on filter papers soaked with the Salkowski reagent (2.0% 0.5 M FeCl₃ in 35% perchloric acid). Colonies of IAA-producers developed a characteristic red halo within the membrane immediately surrounding the colony.

This rapid assay (developed by Bric, J. M, Bostock, R. M., and Silverstone, S. E. 1991. Rapid in-sito assay for indoleacetic acid production by bacteria immobilized on a nitrocellulose membrane. Appl. Environ. Microbiol. 57: 537-538) was positive only for DSM 21663 whose colonies developed characteristic red halos in IAA agar with 0-tryp and 5 mM tryp. Intense red col- or was observed in 5 mM tryp-amended IAA agar plates. A Bacillus strain, B. licheniformis 3086, used for making comparisons, did not show such red halos indicating that it does not produce IAA or produced very little. In our assays, IAA did develop red color even at 0.5 μg applied in 0.5 μl when placed on nitrocellulose membrane directly and exposed to Salkowski reagent. Bric et al., (1991) supra observed that even as little as 0.5 nmoles of IAA can be detected on nitro-cellulose.

### Example 6

### Greenhouse Assays for Pseudomonas DSM 21663

Disease suppression assays: Plant pathogenic bacteria and fungi were obtained from different researchers within the U.S. with authorization/import permits from USDA-APHIS in Washington, D.C.
Reduction of bacterial wilt of tomato: Ralstonia solanacearum causes the devastating bacterial wilt of tomatoes. Two field isolates (AW1, NC251) of the pathogen were provided by Dr Tim Denny of University of Georgia. A small pot experiment was conducted in the greenhouse. Tomato plants of cv. Early Girl were raised in 20 small pots containing Fabard's Potting Mix3B. There were 3 plants per pot. 20 days after planting, when the plants were at 4-leaf stage, DSM 21663 was incorporated into the potting mix in 10 pots by adding 5 ml having 10⁸ cfu/ml. The rest of the plants remained as untreated checks. Plants in five of the DSM 21663-treated pots and 5 pots of the untreated check were inoculated with a syringe containing 1 ml of 10⁶ cfu/ml of Ralstonia solanacearum NC251 a day after the Pseudomonas treatment.

TABLE 7
Suppression of bacterial wilt of tomato
by treatments of Pseudomonas DSM 21663

| | Number of | Number of | | |
|---|---|---|---|---|
| | 20-d old | 27-d old | Number | Percent |
| | plants | plants | of healthy | wilt |
| Treatment | treated | wilted¹ | plants | (%)² |
| Untreated | 15 | 0 | 15 | 0.0 |
| check | | | | |
| DSM21663- | 15 | 0 | 15 | 0.0 |
| treated | | | | |
| Untreated-R. sol | 15 | 13 | 2 | 86.7 |
| inoculated | | | | |
| DSM21663- | 15 | 3 | 12 | 20.0 |
| treated + R. sol. | | | | |
| inoculated | | | | |

¹Tomato plants started wilting in 4-5 d time after inoculation with R. solanacearum.
²DSM 21663 treatment resulted in a ca. 65% reduction in bacterial wilt.

The above greenhouse results are validated from data from a field trial conducted in India by the Indian Institute of Horticultural Research at Bangalore. Pseudomonas DSM 21663 was applied to furrows at 10⁶ cfu/g of wheat bran in a wheat bran formulation before tomato was planted. The wheat bran formulation was prepared in the laboratory from a fermentation broth (1.8 L) of the organism that consisted of 10¹⁰ cfu/ml. The broth was centrifuged at 13,000 rpm for 10 min. The resultant bacterial pellet was re-dissolved in 200 ml of sterile dH₂O and was mixed with 200 g of sterile wheat bran powder. The formulation was dried until the moisture content was 5.0%. DSM 21663 treatment afforded 76% wilt disease control and 115% yield increase. Yield increase is due to its PGPR activity.

TABLE 8
Ralstonia wilt control and yield increases in tomato
(OSAGe Trial No. A07.094). Indian Institute of Horticultural
Research, Bangalore, November 2007 to April 2008

| | Percent | Yield (t/ha) |
|---|---|---|
| | reduction of | (and Percent |
| Treatment, rate and method of application | bacterial wilt | yield increase) |
| Pseudomonas DSM 21663 in Wheat Bran | 76.4 | 18.3 (115.3) |
| @250 g/ha as furrow application | | |
| Streptocycline (300 ppm) seedling root dip | 71.8 | 18.0 (111.8) |
| Untreated control | 0.0 | 0.0 |

In a separate but similar greenhouse experiments, DSM 21663 treatments also suppressed the incidence of:

i) Xanthomonas campestris pv. vesicatoria in tomato (tomato speck)
ii) Pseudomonas syringae pv. phaseolicola in bean (halo blight of bean)

Levels of suppression of tomato speck (development of specks/lesions in plants that received DSM 21663 compared to untreated check) ranged from 45-60% over the untreated control while halo blight of bean was suppressed by >65%. In bean, halo blight incidence was observed only on the primary/cotyledonary leaves in plants that received the DSM 21663 treatments while in the untreated check, halo blight lesions had spread also to the trifoliate leaves that were formed subsequently. These trifoliate leaves also were chlorotic indicating severity of halo blight.

### Growth Enhancement in Tomato

Pilot-plant evaluations and fermentations were carried out for DSM 21663. Four liters of culture containing mid to high 10⁹ cfu/ml were produced for use in greenhouse and field tests. Two batches of wheat bran formulations were made in the laboratory for DSM 21663. These dry wheat bran products (1 and 2) contained 4.6×10⁹ cfu/g, and 3.23×10¹⁰ cfu/g, respectively. This was incorporated into potting mix at 1.23, 0.61 and 0.31 g/gallon pot and tomato (cv. Early Girl) plants raised in the pots were evaluated for growth promotion due to DSM 21663. The data shown below showed that there were significant levels of growth promotion in all three rates of incorporation of DSM 21663.

TABLE 9
Height of tomato (cv. Early Girl) plants that received either
wheat bran (check) or wheat bran formulation of DSM 21663
(Experiment started: May 24, 2007; harvested: Jun. 22, 2007)

| Treatment | Plant Height (cm) | Mean Plant Ht (cm) |
|---|---|---|
| Check (wheat bran | 31.0, 28.5, 26.0, | 32.7 |
| at 1.23 g/gallon pot) | 35.0, 28.0, 29.0, | |
| | 37.0, 39.0, 34.0, | |
| | 39.5 | |
| DSM 21663 in wheat | 39.0, 39.0, 43.0, | 40.0 |
| bran (1) at 10⁹ cfu/g: | 39.0, 40.0 | |
| 1.23 g/gallon pot | | |
| 0.61 g/gallon pot | 42.0, 43.0, 45.0, | 42.0 |
| | 38.0 | |
| 0.31 g/gallon pot | 38.0, 43.0, 41.0 | 40.6 |
| DSM 21663 in wheat | 40.0, 44.5, 40.0, | 39.2 |
| bran(2) at 10¹⁰ cfu/g | 39.0, 33.0 | |
| 0.61 g/gallon pot | | |
| 1.23 g/gallon pot | 47.0, 47.0, 40.0, | 44.0 |
| | 42.0 | |

Wheat bran (1) used at 3 Rates: 1.23 g/gallon pot mix (=250 g (8.8 oz)/cubic yard), 0.61 g/gallon pot mix, and 0.31 g/gallon pot mix.
Wheat bran 2 was used at 2 rates: 0.61 g/gallon pot and 1.23 g/gallon mix.
All of the DSM 21663 treated plants were significantly taller than the untreated check. Growth enhancements due to DSM 21663 were also observed in lettuce and pepper.

### Example 7

### Oxidase Test Assay

The oxidase reaction reflects the ability of a microorganism to oxidize certain aromatic amines, such as tetramethyl-p-phenylene diamine (TPD) (the oxidase reagent), producing colored end products. This is due to the activity of cytochrome oxidase (a.k.a., indophenol oxidase) in the presence of atmospheric oxygen.

Procedure: Using a sterile wooden stick, 2-3 colonies from each strain to be tested were removed and smeared on a piece of filter paper. A drop of the spot test reagent (TPD) was added to each spot. If the organism has oxidase activity, it will turn purple within 10 seconds. An oxidase-negative strain does not turn purple even after 1 min.
Results: Pseudomonas DSM 21663 was oxidase-positive while an authentic strain of Pseudomonas congelans DSM 14929 is oxidase-negative, in repeated tests.
P. corrugata DSM 7228, P. mandelii DSM 17967, and P. mediterranea DSM 11735 were oxidase-positive.

It was concluded that DSM 21663 cannot be a P. congelans isolate.

### Example 8

### Phytotoxicity Test of Volatiles Produced by DSM 21663

### Partition Plate (1-Plate) Assays for Phytotoxicity:

To observe changes in the growth response of plant and algal tissues exposed to volatiles produced by Pseudomonas spp. DSM 21663, I-plates were used. The partition in the middle will prevent the physical movement of bacteria from one half of the plate to the other. The I-plates were prepared with Standard Methods Agar (SMA) (10 ml) in one half. The opposite half was layered with a moistened sterile filter paper (Whatman No. 1). In untreated control plates, the filter paper received 10 seeds of either wheat, or tomato or lettuce or Poa spp (weed) while the SMA remained blank. In DSM 21663-treated plates, opposite to the seeds, 20 μl of freshly grown culture of DSM 21663 that contained 10⁸ cfu/ml was added to the SMA. The plates were wrapped with para-film and were incubated in the dark for 5 days.

Result: The number of germinated seeds in control and treatments showed that seeds of wheat, tomato, lettuce and Poa spp. exposed to DSM 21663 failed to germinate. In untreated control, 100% seed germination was observed.
Similar I-Plate Assays were used to test phytoxicity of DSM 21663 volatiles to algae. Six species of algae were used: Anabaena, Lyngbya, Oedogonium, Oscillatoria, Spirogyra, and Volvox. In I-Plates, opposite to 20 μl of DSM 21663, the algae were grown in 10 ml of Algal-Gro (liquid medium) and incubated on the laboratory bench/growth chamber for 14 days. Three replications were maintained for each species studied. Growth of algae was scored in untreated control and DSM 21663 treated assay plates using a 1 (least amount of growth)-to-5 (maximum growth).
Result: Average scores for algal growth was 5 for untreated Lyngbya, Anabaena, Oscillatoria and Spirogyra while these scores were 0 for DSM 21663-treatment of Lyngbya, Anabaena, Oscillatoria and was 1 for Spirogyra.

### Insect Toxicity Assays:

Wingless mutants of Drosophila malanogaster (fruit fly) were obtained from Carolina Biologicals. Twenty insects were exposed either to blank SMA contained at the bottom of a test tube or SMA inoculated with 20 μl of a fresh culture of DSM 21663 that contained 10⁸ cfu/ml. A sterile pad of cheese cloth (10 mm thick) separated the insects and the agar. The number of insects that remained alive after 16, 24, 40 and 48 hrs was counted both in control and DSM 21663 treatments. Two experiments were conducted.

Results: In the first experiment, 21% of insects died after 24 hrs in the untreated control while 84% of insects died in DSM 21663 treatment. In experiment 2 results were recorded after 48 hrs when 64% of insects had died in untreated control while 93% had died in DSM 21663 treatments.

### Example 9

### Test of Ability of DSM21663 to Inhibit Germination of sclerotia Derived from the Plant Pathogen Sclerotinia sclerotiorum

Sclerotia are the resting bodies originating from the pathogen Sclerotinia sclerotiorum and which is the cause of the disease known as white mold on a wide variety of broadleaf crops. In the present example we investigated the ability of volatiles produced from DSM21663 to inhibit germination of sclerotia. The inhibition of DSM21663 was compared to a negative control.
Treatments (replicated 4 times) were:

DSM21663

Control (no bacteria was streaked)

### Experimental Design:

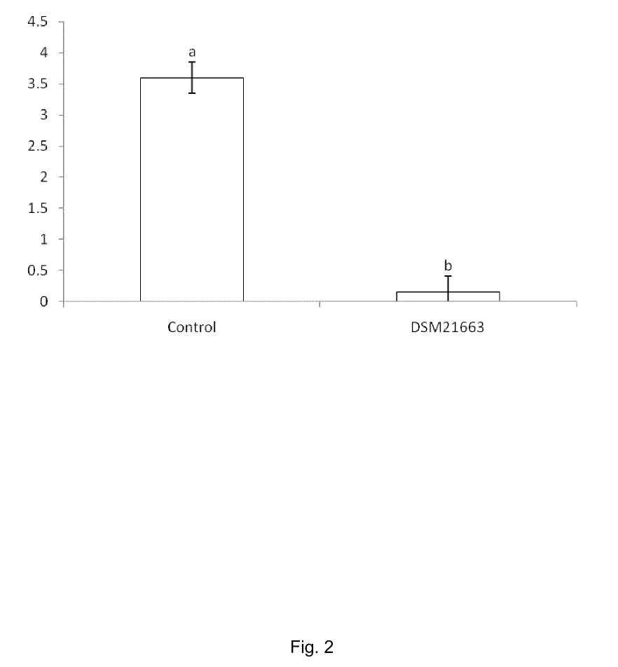

A three compartment Petri plate was used. The first compartment had TSA media (Tryptic Soy Agar from BD Company, USA) streaked with the bacterial strain, the second compartment had PDA media (Potato Dextrose Agar from EMD Chemicals Inc., Germany) inoculated with surface sterilized sclerotia, and the third compartment was left empty.
The results are given in FIG. 2 and in the table below. FIG. 2 shows the influence of volatiles produced by the bacterium according to the invention on mycelial growth after 3 days. DSM21663 showed a clear inhibitory effect.

TABLE 10
Sclerotial germination after 10 days:
germinated(+)/non-germinated (−).

Sclerotial germination

| Treatment | Rep1 | Rep2 | Rep3 | Rep4 |
|---|---|---|---|---|
| DSM21663 | + | − | − | − |
| Control | + | + | + | + |

DSM21663 had the highest impact on sclerotial myceliogenic germination.

In another experiment a slightly different experimental design was used as described below. The effect of volatiles produced from DSM21663 was compared to two other species of Pseudomonas known to have inhibitory effects on plant pathogens.
Treatments (replicated 6 times) were:
DSM21663
Ps 142 (P. fluorescens) Ps 145 (P. putida)
Control (no bacteria)
   Design: Two experimental designs were used:
Bacteria were streaked on TSA in the bottom dish of a Petri plate. One surface sterilized sclerotium was placed in the centre of the bottom dish of a second Petri plate containing PDA. The dish containing the sclerotium was inverted over the TSA plate and sealed tightly with Para-film.
   The control plates had no bacteria. Plates were incubated for 7 days in the dark at room temperature. After 7 days, all sclerotia were removed from the plates and tested for viability in a fresh Petri plate. The results are given in the tables below.

TABLE 11
Sclerotia germination when bacteria were streaked on
TSA: germinated(+)/non-germinated (−).

| | Treatment | | | | | |
|---|---|---|---|---|---|---|
| | Rep1 | Rep2 | Rep3 | Rep4 | Rep5 | Rep6 |
| DSM21663 | − | − | − | − | − | − |
| P. putida | − | − | − | − | − | − |
| P. fluorescens | − | − | − | + | + | + |
| Control | + | + | + | + | + | + |

### Example 10

Biocontrol of Fusarium Head Blight of Wheat Under Growth Chamber Conditions

### Experiment:

1. Positive control (heads were sprayed with a suspension of Fusarium conidia in a conc. of 7×10⁴1 ml)
2. Negative control (sprayed with water only)
3. Proline chemical control (heads were inoculated with Proline and Fusarium conidia)
4. Ba146 (Bacillus subtilis ATCC 202152 disclosed in U.S. Pat. No. 6,896,883) (heads were inoculated with a suspension of Ba 146 applied at 1.3×10⁷/ml and Fusarium conidia at 7×10⁴/ml)
5. Ps146 (Pseudomonas DSM21663) (heads were inoculated with a suspension of Ps146 applied at 1.3×10⁷/ml and Fusarium conidia at 7×10⁴/ml)

All biocontrol (Ba146 and Ps146) and chemical (Proline) treatments were applied over wheat heads 24 hours before Fusarium inoculations. The rate of application was 1 ml per 2 wheat heads. After inoculations with Fusarium conidia all plants were exposed to high humidity conditions for 50 hours. Disease ratings were done 10 days after the pathogen was applied.

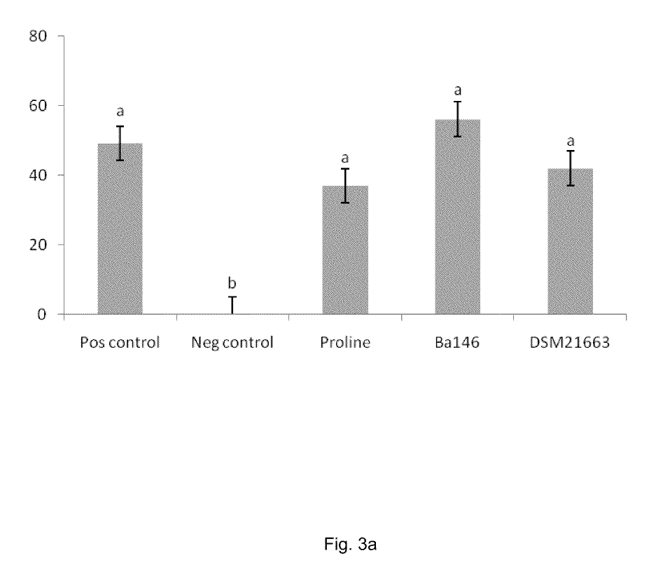

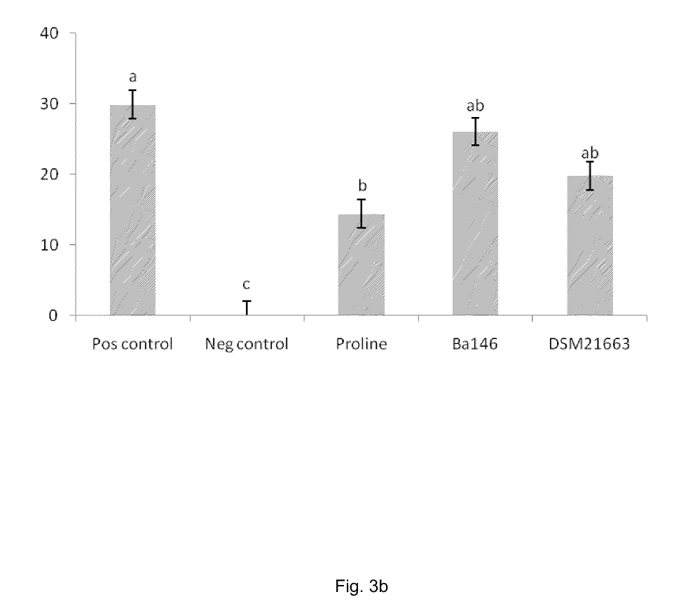

Disease incidence as the count of infected heads divided by the total number of heads from the replicate and is shown in FIG. 3a (N=5, P=0.05, student T test). Infected heads severity as average of severity scores from infected heads (based on disease scale) is shown in FIG. 3b (N=5, P=0.05, student T test).

The data indicate that the isolate according to the invention performs at a similar level as the chemical control in biocontrol of Fusarium Head Blight.

### Deposit of Biological Material

The following biological material has been deposited under the terms of the Budapest Treaty with the Deutsche Sammlung von Mikroorganismen and Zellkulturen GmbH (DSMZ), Inhoffenstr. 7B, D-38124 Braunschweig, Germany, and given the following accession number:

| Deposit | Accession Number | Date of Deposit |
|---|---|---|
| Pseudomonas spp | DSM 21663 | 17 Jul. 2008 |

The strain has been deposited under conditions that assure that access to the culture will be available during the pendency of this patent application to one determined by foreign patent laws to be entitled thereto. The deposit represents a substantially pure culture of the deposited strain. The deposit is available as required by foreign patent laws in countries wherein counterparts of the subject application, or its progeny are filed. However, it should be understood that the availability of a deposit does not constitute a license to practice the subject invention in derogation of patent rights granted by governmental action.

1. An isolated Pseudomonas bacterium which has at least the characteristics:
i) is fluorescent;
ii) naturally encodes for the production of indophenol oxidase;
iii) naturally encodes for the production of 2,4-diacetylphloroglucinol, pyrrolnitrin, and indole-3-acetic acid;
iv) has the ability to promote solubilization of soil phosphate;
v) suppresses soil-borne plant pathogens of both fungal and bacterial origin; and
(vi) has a 16S rDNA nucleic acid sequence comprising SEQ ID No. 1 or a nucleic acid sequence at least 98% identical to SEQ ID No. 1.

2. The Pseudomonas bacterium according to claim 1, where the pathogens comprise the fungi Pythium, Phytophthora, Rhizoctonia, Sclerotinia, Venturia and Fusarium.

3. The Pseudomonas bacterium according to claim 1, where the pathogens comprise the bacteria Ralstonia, Xanthomonas, Pseudomonas and Erwinia.

4. The Pseudomonas bacterium according to claim 1, which is capable of producing gaseous volatiles with phytotoxicity towards fungi, algae, plants or insects.

5. The Pseudomonas bacterium according to claim 4, wherein the insect is Drosophila.

6. An isolated Pseudomonas strain, where the said strain is DSM 21663.

7. A composition comprising the isolated Pseudomonas bacterium according to claim 1, and a carrier.

8. A method of enhancing plant growth, comprising applying to plants, plant seeds, or soil surrounding plants, or plant seeds a composition according to claim 7.

9. The method according to claim 8, wherein soil-borne root and foliar pathogens are suppressed.

10. The method according to claim 8, wherein availability of phosphorous for plant uptake is improved.

11. The method according to claim 10, wherein the phosphorous is from a source originally present in the soil, sources added to the soil, and combinations thereof.

12. The method according to claim 8, wherein the plant is selected from the group consisting of alfalfa, rice, wheat, barley, rye, cotton, sunflower, peanut, corn, potato, sweet potato, bean, pea, chicory, lettuce, endive, cabbage, brussel sprout, beet, parsnip, turnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane.
